(19) United States
(12) Patent Application Publication
(10) Pub. No.: US 2004/0076585 A1
(43) Pub. Date: Apr. 22, 2004

(54) Nanoparticles of cyclic tetrapyrrolic compounds as gene and drug delivery carriers

(76) Inventor: Xianchang Gong, San Diego, CA (US)

Correspondence Address: Xianchang Gong, 9505 Genesee Ave., Unit 524, San Diego, CA, 92121, US

(21) Appl. No.: 10/679,730

(22) Filed: Oct. 6, 2003

Related U.S. Application Data
(60) Provisional application No. 60/418,892, filed on Oct. 16, 2002.

(51) Int. Cl. A61K 49/00; A61K 31/555

(52) U.S. Cl. 424/9.61; 514/185; 514/410

(57) ABSTRACT

The present invention relates to a method using nanoparticles of cyclic tetrapyrrolic compounds as gene and drug delivery agents. Pharmaceutical agents can be packed or condensed inside nanoparticles of cyclic tetrapyrrolic compounds and delivered to cells. In vitro experiments showed nanoparticles of cyclic tetrapyrrolic compounds can be effectively delivered into cells.

### FIELD OF THE INVENTION

The present invention relates generally to compositions and methods of using nanoparticles of cyclic tetrapyrrolic compounds as gene delivery and drug delivery carriers. More specifically, the invention relates to compositions and methods of using nanoparticles of cyclic tetrapyrrolic compounds to delivery DNA sequence, RNA sequence, peptide, protein, or non-soluble drug molecules into cells in vivo.

### REFERENCE CITED

Gong, X. “Method to prepare porphyrin nanoparticles and its application as oxidation catalyst”, USPTO10/163119

Gong, X.; Tatjana, M.; Xu, C.; Batteas, J.; Drain, C. M. “Preparation and characterization of porphyrin nanoparticles”, J. Am. Chem. Soc.; 2002, 124, 14290-14291.

Mountain, A. “Gene Therapy: The First Decade”. Trends Biotechnol. 2000, 18, 119-128.

Felgner, P. L. “Particulate systems and Polymers for in vitro and in vivo Delivery of Polynucleotides”. Adv. Drug Delivery Rev. 1990, 5, 163-187.

Behr, J. P. “Gene Transfer with Synthetic Cationic Amphiphiles: Prospects for Gene Therapy”. Bioconjugate Chem. 1994, 5, 382-389.

Littler, B. J.; Ciringh, Y.; Lindsey, J. S.; “Investigation of Conditions Giving Minimal Scrambling in the Synthesis of trans-Porphyrins from Dipyrromethanes and Aldehydes”, J. Org. Chem.; 1999; 64(8); 2864-2872.

Drain, C. M.; Gong, X.; Ruta, V.; Soll, C. and Chicoineau, P. “Combinatorial synthesis of functional libraries: Identification of New, Amphipathic Motifs for Biomolecule Binding”, J. Combin. Chem., 1999, 1, 286-290.

Hunter, C. A.; Sanders, J. K. M. “The nature of pi-pi interactions”, J. Am. Chem. Soc. 1990, 112, 5525-34

Bonnet, R. “Photosensitizers of the porphyrin and phthalocyanine series for photodynamic therapy”, Chem. Soc. Rev. 1995,19-32

### BACKGROUND

Nanoparticles composed of polymers, liposomes and virus vectors have been extensively studied as gene delivery agents (Mountain, A. “Gene Therapy: The First Decade”. Trends Biotechnol. 2000, 18, 119-128; Felgner, P. L. “Particulate systems and Polymers for in vitro and in vivo Delivery of Polynucleotides”. Adv. Drug Delivery Rev. 1990, 5, 163-187; Behr, J. P. “Gene Transfer with Synthetic Cationic Amphiphiles: Prospects for Gene Therapy”. Bioconjugate Chem. 1994, 5, 382-389.) The advantages and disadvantages of these gene delivery methods have been well reviewed. Gene therapy has promising applications in the treatment of genetic disorders such as many forms of cancer and chronic infectious diseases such as allergy and AIDS.

Cyclic tetrapyrrolic compounds include porphyrins and reduced porphyrins such as chlorins. Porphyrins and chlorins have been known for a long time to accumulate inside tumor cells. This property of porphyrins and chlorins may help the nanoparticles composed of porphyrins or chlorins accumulate inside the tumor cells. By controlled releasing of the therapeutic agents, the therapeutic agents may specifically remained inside tumor cells, so the efficiency of the therapeutic agents could be dramatically improved.

The present invention relates to compositions and methods of using nanoparticles of cyclic tetrapyrrolic compounds for gene delivery and drug delivery. More particularly, this invention relates to compositions and methods for use, and making thereof, for delivering nucleic acids as gene therapy applications or other non-soluble bioactive molecules such as protein, peptides or small non-soluble drugs.

### SUMMARY OF THE INVENTION

The present invention relates to a method using nanoparticles of cyclic tetrapyrrolic compounds as gene delivery or drug delivery agents. A composition for use as a gene delivery carrier is comprising of nanoparticles of a cyclic tetrapyrrolic compound and a nucleic acid. A method of delivering a nucleic acid into a cell in vivo comprising the steps of: a. mixing the nanoparticles of a cyclic tetrapyrrolic compound with a selected nucleic acid; b. Contacting the selected cell with the complex under conditions to maintain the viability of the cell.

### BRIEF DESCRIPTION OF THE DRAWINGS

FIG. 1. DSL characterization of Fe-tetrakis[di(ethylene glycol)monomethyl-2-pyridium]porphyrin pentachloride nanoparticles

FIG. 2. AFM images of Fe-tetrakis[di(ethylene glycol)monomethyl-2-pyridium]porphyrin pentachloride nanoparticles on glass in air

FIG. 3. Histograms of Fe-tetrakis[di(ethylene glycol)monomethyl-2-pyridium]porphyrin pentachloride nanoparticles on glass in air taken by topographic AFM measurements.

FIG. 4. UV-Vis. Spectra of 5,10,15,20-Tetrakis(4-carboxyl)porphyrin in DMASO (a) and its nanoparticles in water (b).

FIG. 5. Cells intake of nanoparticles of 5,10,15,20-Tetrakis(4-methoxyphenyl)porphyin. Top: control (without porphyrin nanoparticles); Bottom: with porphyrin nanoparticles.

FIG. 6. Cells intake of nanoparticles of 5,10,15,20-Tetrakis(pentafluorphenyl)porphyin. Top: control (without porphyrin nanoparticles); Bottom: with porphyrin nanoparticles.

### DETAILED DESCRIPTION OF THE INVENTION

The present invention related to compositions and methods of using nanoparticles of cyclic tetrapyrrolic compounds as gene delivery and drug delivery agents. As used herein, “cyclic tetrapyrrolic compound” referred to a class of organic molecules that have four pyrrole units in its ring system. Particularly in this invention, cyclic tetrapyrrolic compounds are porphyrins or reduced porphyrins such as chlorin and bacteria chlorine.

Non-viral methods for gene delivery have several advantages over virus vectors. that arise largely from the fact that only the desired genes are delivered. But the problem with non-viral gene delivery methods is the low transfection efficiency compared to virus vectors. In order to overcome this problem, extensive studies have been carried out on the synthesis of new lipids with different functional groups, and on the design and synthesis of new polymeric systems.

Porphyrins have been known for a long time to accumulate in cells, and in some cases preferentially inside tumor cells. Thus it is reasonable to expect that porphyrin nanoparticles-DNA complex may accumulate inside tumor cells as well. Since porphyrins have 4-8 sites to attach various chemical moieties, some sites can derivatized to target the compound towards a given cell type while others can bear cationic groups designed to interact with DNA or RNA. An additional feature of this system is that the fluorescence of the macrocycle will serve as an indicator of incorporation into the cell. This will be a great advantage for using porphyrin nanoparticles as gene delivery carriers due to the porphyrin nanoparticles-DNA complex can target specific cells such as tumor cells. This “self-targeting” property will eliminate the need to add an additional targeting agent in the nanoparticles in order to achieve higher gene transfection efficiency.

Compared to the existent carriers for gene delivery, porphyrin nanoparticles represent a novel approach. There are two possible structures of the porphyrin nanoparticles-DNA complex. One possible structure is like a ‘sandwich’ with DNA condensed between the layers of porphyrin molecules. Another possible structure of the porphyrin-DNA complex is like an ‘egg’ with DNA condensed in the center—essentially a porphyrin nanoparticle with a DNA center. The process of the porphyrin nanoparticles-DNA complex entering cells and mechanism of DNA escaping from the aggregate may be by endocytosis and endosomal escape.

Heuristically, there are three different intermolecular interactions between porphyrins and RNA or DNA, namely electrostatic forces, hydrogen binding, and Van der Waals forces. The driving force for porphyrins to condense DNA could be a combination of these three different interactions. In addition to the polycationic porphyrin serving as a counter ion to the polyanionic phosphate backbone, and more effectively screening the charge than the normal monovalent cations found in vivo, previous studies show porphyrins with suitable substitutents can fit well between the loops of DNA, and intercolate within the DNA (Drain, C. M.; Gong, X.; Ruta, V.; Soll, C. and Chicoineau, P. “Combinatorial synthesis of functional libraries: Identification of New, Amphipathic Motifs for Biomolecule Binding”, J. Combin. Chem., 1999, 1, 286-290). These phenomena may facilitate the DNA condensing process. Another driving force for the condensation of DNA inside poprhyrin nanoparticles may be the strong π-stacking effects between porphyrin molecules(Hunter, C. A.; Sanders, J. K. M. “The nature of pi-pi interactions”, J. Am. Chem. Soc. 1990, 112, 5525-34), which may serve to enhance the cooperative formation of the nano aggregate. The transfection efficiency of gene delivery can be improved by adjusting the π-stacking effect between porphyrin molecules and by varying the substitutes on the porphyrin ring.

Porphyrins accumulate in tumors through mechanisms that are not well understood(Bonnet, R. “Photosensitizers of the porphyrin and phthalocyanine series for photodynamic therapy”, Chem. Soc. Rev. 1995,19-32), but may include their amphipathic solubility properties which translates to an affinity to lipoprotein, especially low-density lipoproteins (LDLs). Rapidly dividing cell, healthy or unhealthy, have more receptors for LDLs than do quiescent cells, porphyrin may piggybacks onto LDL, and the LDL and its passenger are picked up by a receptor. This may explain the preferential accumulation of porphyrins in rapidly dividing cells, such as the endothelial cells in tumors and new blood vessels. Similarly, the porphyrin nanoparticles-DNA complex may bind to LDL through designed chemical moieties on the porphyrin molecule, thus on the particle surface.

The mechanism of DNA condensation inside porphyrin nanoparticles may be different than other kinds of gene delivery carriers, such as cationic lipids and polymers. The weak van der Waal's forces of the former tend to result in liposomes. A given polycationic polymer will cooperatively bind DNA but the polymers have little affinity for each other. The driving forces for DNA condensation by porphyrins may due to both the interactions between porphyrins and interactions with DNA—both of which can be modulated by chemical modification.

Structures of the cyclic tetrapyrrole compounds used to prepare nanoparticles in this invention are showing below:

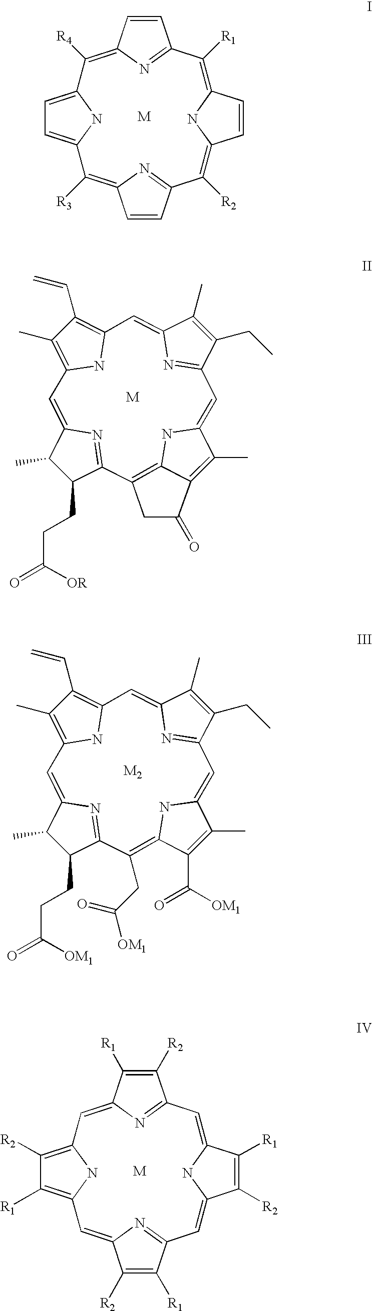

Wherein:

In formula I, R₁, R₂, R₃ and R₄ are substitutes on the porphyrin ring and M is 2H⁺ or a metal ion selected from ions of a group of metals consisting of Mg, Fe, Mn, Co, Ni, Cu, Zn, Sn, Cr, V, Ru, Pt or Pd.

In formula II, R is a substitute and M is 2H⁺ or a metal ion selected from ions of a group of metals consisting of Mg, Fe, Mn, Co, Ni, Cu, Zn, Sn, Cr, V, Ru, Pt or Pd.

In formula III, M₂ is 2H⁺ or a metal ion selected from ions of a group of metals consisting of Mg, Fe, Mn, Co, Ni, Cu, Zn, Sn, Cr, V, Ru, Pt or Pd. M₁ is H⁺ or a metal ion selected from ions of a group of metals consisting of Li, Na, or K.

In formula IV, R₁ and R₂ are substitutes and M is 2H⁺ or a metal ion selected from ions of a group of metals consisting of Mg, Fe, Mn, Co, Ni, Cu, Zn, Sn, Cr, V, Ru, Pt or Pd.

In some embodiments, R in the formula II or III is

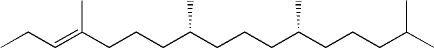

In some embodiments, the cyclic tetrapyrrole compounds used to prepare nanoparticles in this invention includes compounds having the formula V, VI or VII:

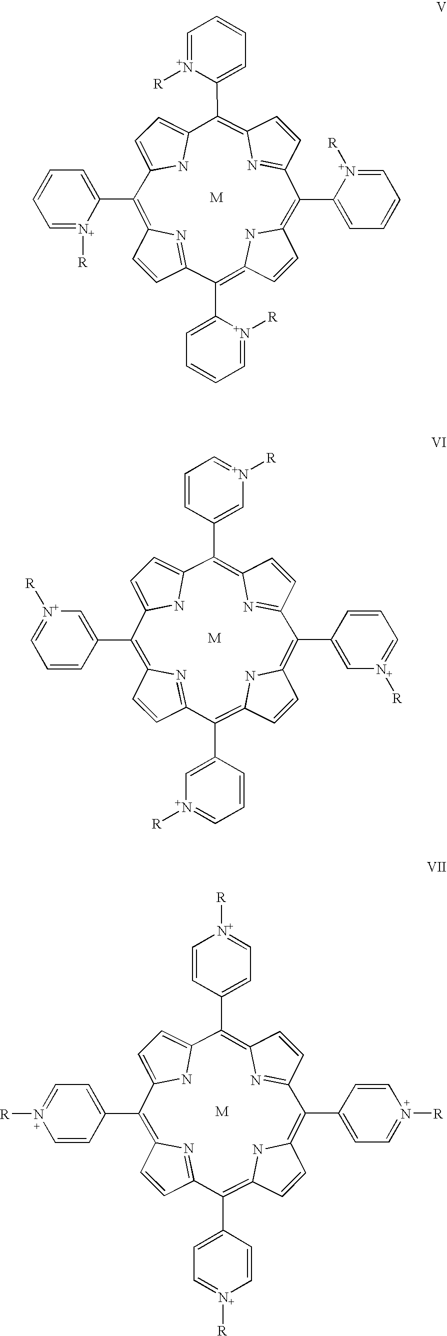

Wherein: R is substitute on the porphyrin ring and M is 2H⁺ or a metal ion selected from ions of a group of metals consisting of Mg, Fe, Mn, Co, Ni, Cu, Zn, Sn, Cr, V, Ru, Pt or Pd.

In some embodiments, R in the formula V, VI and VII is —CₙH₂ₙ₊₁, or —(CₘH₂ₘ)ⱼ(OCₘH₂ₘ)ₚOR where in n, m, j, p=1-10

In this invention, ‘gene’ referred to DNA or RNA sequence, ‘drug’ referred to peptide, protein, or non-soluble drug molecules.

Nanoparticle preparation: Nanoparticles of cyclic tetrapyrrolic compounds can be prepared by mixing solvent techniques in high yield and narrow distribution of particle size, see Gong, X., U.S. patent application Ser. No. 10/163,119, filed Jun. 5, 2002 and Gong, X.; Tatjana, M.; Xu, C.; Batteas, J.; Drain, C. M. “Preparation and characterization of porphyrin nanoparticles”, J. Am. Chem. Soc.; 2002, 124, 14290-14291.

Loading of genes into the nanoparticle: Genes can be condensed or packed inside the nanoparticles of cyclic tetrapyrrole compounds by premixing genes and cyclic tetrapyrrole compounds before nanoparticles formation.

Loading of bioactive molecules into the nanoparticle: Bioactive molecules can be condensed or packed inside the nanoparticles of cyclic tetrapyrrole compounds by premixing them with cyclic tetrapyrrole compounds before nanoparticles formation.

### EXAMPLES

The following chemicals were utilized in examples 1-16 that follow. 5,15-di(4-bromophenyl)-10,20-di(mesityl)porphyrin was prepared according to published method (Littler, B. J.; Ciringh, Y.; Lindsey, J. S.; “Investigation of Conditions Giving Minimal Scrambling in the Synthesis of trans-Porphyrins from Dipyrromethanes and Aldehydes”, J. Org. Chem.; 1999; 64(8); 2864-2872). All other chemicals were purchased from Aldrich. DLS experiments were performed with a PD2000DLS (Precision Detector). AFM experiments were performed with Nanoscope IIIa Multi-probe microscope (Digital Instruments). All measurements were made in air at room temperature using standard Si₃N₄ tips (Veeco) with nominal tip radius of curvature 20-60 nm and spring constant of 0.12 N/m. UV-Vis absorption spectra were obtained on a Carey 1 spectrophotometer.

### Example 1

### Synthesis Nanoparticles of Fe-tetrakis[di(ethylene glycol)monomethyl-2-pyridium]porphyrin pentachloride

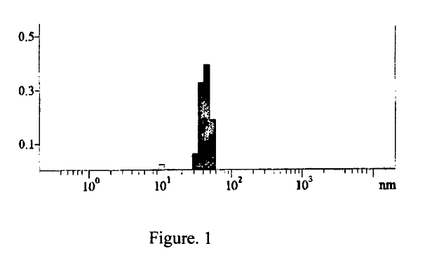

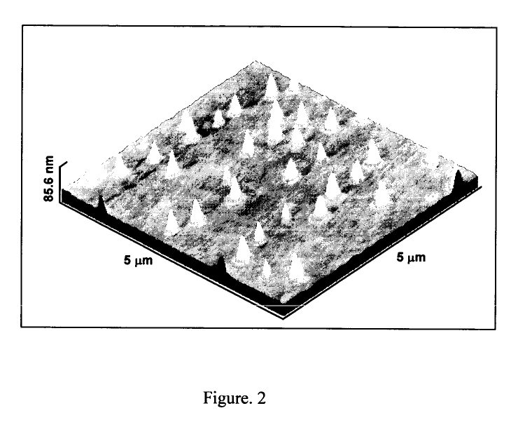

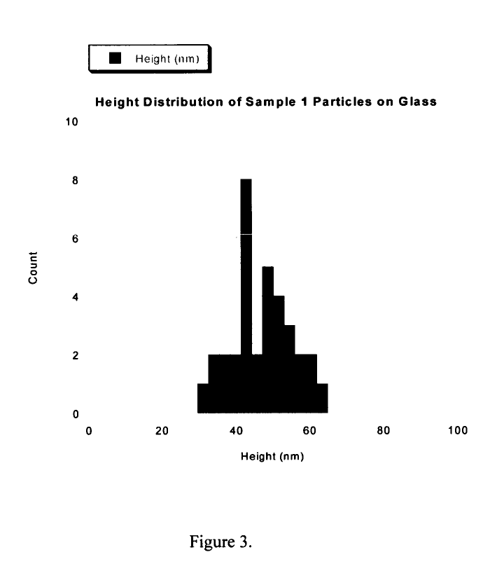

1 mg Fe-tetrakis[di(ethylene glycol)monomethyl-2-pyridium]porphyrin pentachloride was dissolved in 50 ul water followed by adding 5 ml acetonitrile. Yield: >95%. Average particle radius measure by DSL: 43.9 nm, FIG. 1. Sizes and distributions of the nanoparticles measured by AFM are in the range of 10 to 70 nm, FIG. 2. Histograms of nanoparticles on glass in air taken by topographic AFM measurements, FIG. 3.

### Example 2

### Synthesis Nanoparticles of 5,10,15,20-Tetrakis(4-carboxyl)porphyrin

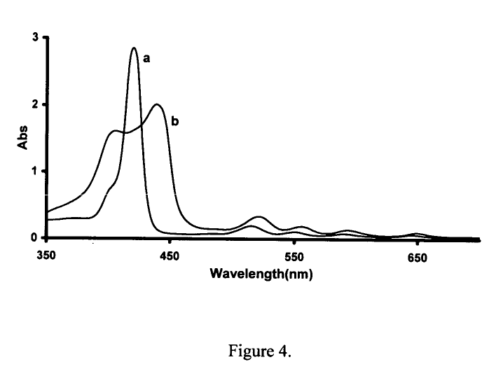

1.9 mg 5,10,15,20-Tetrakis(4-carboxyl)porphyrin was dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 46.6 nm. UV-Visible spectrum of porphyrin nanoparticles is clearly different compared to porphyrin in solution, FIG. 4.

### Example 3

### Synthesis Nanoparticles of 5,10,15,20-Tetrakis(4-methoxycarbonylphenyl)porphyin

1.0 mg 5,10,15,20-Tetrakis(4-methoxycarbonylphenyl)porphyin was dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 58.2 nm.

### Example 4

### Synthesis Nanoparticles of 5,10,15,20-Tetrakisphenylporphyin

3.0 mg 5,10,15,20-Tetrakisphenylporphyin was dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 11.3 nm.

### Example 5

### Synthesis Nanoparticles of 5,10,15,20-Tetrakis(4-methoxyphenyl)porphyin

1.5 mg 5,10,15,20-Tetrakis(4-methoxyphenyl)porphyin porphyrin was dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 34.1 nm.

### Example 6

### Synthesis Nanoparticles of 5,10,15,20-Tetrakis(4-pyridyl)porphyin

0.7 mg 5,10,15,20-Tetrakis(4-pyridyl)porphyin was dissolved in 4 ml pyridine to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 70 nm.

### Example 7

### Synthesis Nanoparticles of 5,15-di(4-bromophenyl )-10,20-di(mesityl)porphyrin

1.6 mg 5,15-di(4-bromophenyl )-10,20-di(mesityl)porphyrin was dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 24 nm.

### Example 8

### Synthesis Hybrid Nanoparticles of 5,10,15,20-Tetrakisphenylporphyin and 5,10,15,20-Tetrakis(4-methoxyphenyl)porphyin

1.2 mg 5,10,15,20-Tetrakisphenylporphyin and 1.6 mg 5,10,15,20-Tetrakis(4-methoxyphenyl)porphyin were dissolved in 4 ml DMSO to make the stock solution.

0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 37.4 nm.

### Example 9

### Synthesis Nanoparticles of 5,10,15,20-Tetrakisphenylporphyin Iron(III) chloride

6 mg 5,10,15,20-Tetrakisphenylporphyin Iron(III) chloride was dissolved in 0.6 ml CH₂Cl₂ in a test tube followed by adding 50 ul Triethylene glycol monomethylether. After 1 minute, 5 ml water was added to this mixture and a glass rod was used to stir the mixture. Yield: >99%. Average particle radius measure by DSL: 12 nm.

### Example 10

### Synthesis Nanoparticles of 2,3,7,8,12,13,17,18-Octaethylporphyrin magnesium(II)

0.6 mg 2,3,7,8,12,13,17,18-Octaethylporphyrin magnesium(II) was dissolved in 0.4 ml DMSO in a test tube followed by adding 50 ul NH₂(CH₂)₃NH(CH₂)₃NHC₁₁H₂₃. After 1 minute, 5 ml water was added to this mixture and a glass rod was used to stir the mixture. Yield: >99%. Average particle radius measure by DSL: 4.3 nm.

### Example 11

### Synthesis Nanoparticles of Chlorophyll a

0.6 mg Chlorophyll a was dissolved in 0.4 ml DMSO in a test tube followed by adding 50 ul triethylene glycol monomethylether. After 1 minute, 5 ml water was added to this mixture and a glass rod was used to stir the mixture. Yield: >99%. Average particle radius measure by DSL: 22 nm.

### Example 12

### Synthesis nanoparticles of 5,10,15,20-Tetrakis(pentafluorphenyl)porphyin

5 ml water was added to a mixture of 400 ul 0.745 mM 5,10,15,20-Tetrakis (pentafluorphenyl)porphyin solution in DMF and 20 ul triethylene glycol monomethylether. A glass rod was used to stir the mixture. Yield: >99%. Average particle radius measure by DSL: 77 nm.

### Example 13

### Synthesis Nanoparticles of 5,10,15,20-Tetrakis(2-pyridium)porphyrin Fe (III) chloride

5 ml water was added to a mixture of 50 ul 5 mM 5,10,15,20-Tetrakis(2-pyridium)porphyrin Fe (III) chloride solution in water and 60 ul hexaethyleneglycol methyl ether to make nanoparticles. Yield: >99%. Average particle radius measure by DSL: 174 nm.

### Example 14

### Synthesis of Fe-tetrakis[di(ethylene glycol)monomethyl-2-pyridium]porphyrin pentachloride

Step 1: 10.6 g 2-pyridinecarboxaldehyde and 6.7 g pyrrole were added to 1000 ml acetic acid in a 2 L flask. The mixture was refluxed for 2 hours. The solvent was removed under vacuum and the crude product was loaded onto a flash silica gel column. The column was developed by CHCl₃. 0.51 g pure 5,10,15,20-Tetrakis(2-pyridyl)porphyin was obtained.

Step 2: 24 g diethylene glycol monomethylether and 30 g triethylamine were added to 1000 ml CH₂Cl₂. The mixture was cooled on ice. 38 g p-toluenesulfonyl chloride was added to the mixture. The mixture was stirred overnight at room temperature. The reaction mixture was cooled on ice for 3 hours to precipitate out the triethylamine hydrochloride salt. After filtration, the solvent was removed under vacuum to obtain the desired product. 30 g diethylene glycol monomethylether tosylate was made.

Step 3: 0.51 g pure 5,10,15,20-Tetrakis(2-pyridyl)porphyin, 30 g diethylene glycol monomethylether tosylate and 1 g FeCl₃ were added to 10 ml N,N-Dimethylformamide. The reaction was took place for 12 hours at 90° C. After the reaction was finished, water was added to extract the final product. 0.4 g Fe-tetrakis[di(ethylene glycol)monomethyl-2-pyridium]porphyrin pentachloride was made. UV-Visible spectrum in water: 407, 584. ESI-MS: 217[(M−5Cl⁻)⁵⁺/5].

### Example 15

### In Vitro Delivery of Porphyrin Nanoparticles Into Cells

Cell Culture Cells were maintained in Dulbecco's Modified Eagle Medium (DMEM) (GIBCOBRL), 10% bovine calf serum (HyClone), 1% antimycotic (GIBCOBRL) at 37° C. and in 5% CO₂ atmosphere, or otherwise stated. For experiments, 2×10⁵ cells/mL were seeded in cell culture plates and then allowed to grow for 24 hours. For all the experiments involve porphyrins, the drug was added to the cells 24 hours prior to experiments to allow maximum taken up by the cells.

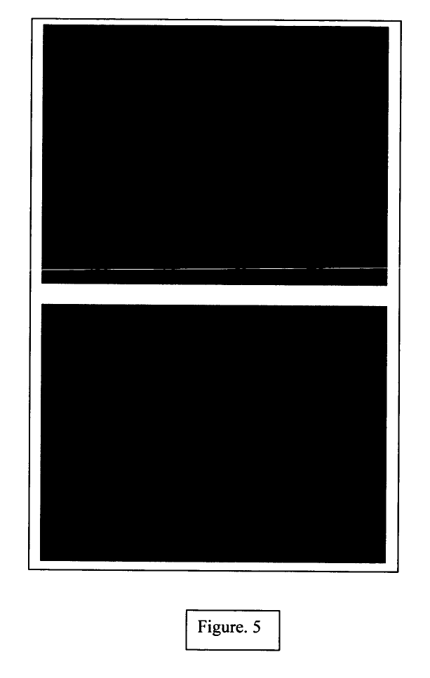

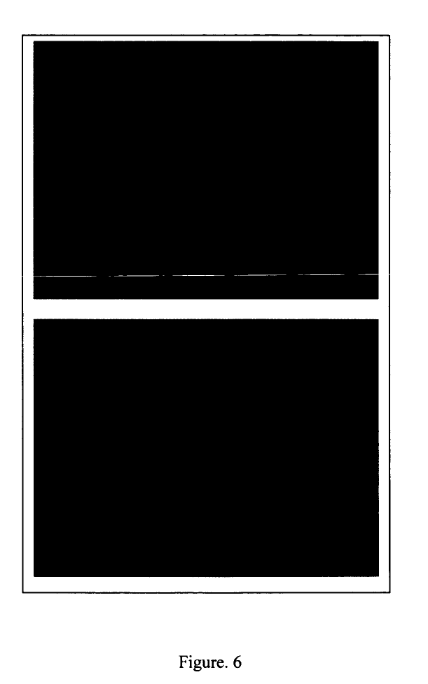

Fluorescent Imaging Cells were plated onto cover slips in cell culture dishes. Porphyrins nanoparticles were made into the growth medium. Cells were washed twice with PBS (136 mMNaCl, 2.6 mM KCl, 1.4 mMKH₂PO₄, 4.2 mM Na₂HPO₄), fixed in 4% paraformaldehyde in PBS for 20 min at room temperature, and then washed with PBS 5 times. The cover slips were mounted in Dako fluorescent mounting medium, put onto slides, air dried, and then visualized using a Nikon Optiphot 2 fluorescent microscope. (Excitation: 505-565 nm and Emission: 565-685 nm). For comparison, images were also captured under phase contract light microscope. Data were saved and images are presented as the raw data. Results of cells intake of nanoparticles of 5,10,15,20-Tetrakis(4-methoxyphenyl)porphyin are shown in FIG. 5; Results of cells intake of nanoparticles of 5,10,15,20-Tetrakis(pentafluorphenyl)porphyin are shown in FIG. 6.

### Example 16

### In Vitro Gene Transfection

MDA231 (human breast cancer cell line) cells were plated onto cover slips in cell culture dishes. Cells were cultured in DMEM (Dulbecco's Modified Eagle Medium plus 10% bovine calf serum and 1% antimycotic), incubated at 37° C., 10% CO₂. A minimum time period of overnight was allowed for the cells to be attached well to the cover slips. 1 mg Fe-tetrakis[di(ethylene glycol)monomethyl-2-pyridium]porphyrin pentachloride was dissolved in 50 μl water to make a stock solution. 3 μl stock solution was transferred to mix with 3 μg pEGFP-c1 plasmid (contains GFP gene), and incubated for 24 h in the dark at room temperature to form the porphyrin nanoparticles-DNA complex. 500 μl Serum-free medium was added to this mixture 20 min prior to the addition to the cell culture. The complete medium in culture dish was replaced with 2 ml serum-free medium, when cells reached 50-90% confluence. The porphyrin-DNA mixture was added to the cells and incubated at 37° C., 10% CO₂ for 3 h. Serum-free medium was changed back to 3 ml complete medium. Cells were incubated at normal condition for 60 h and were washed twice with PBS, fixed in 3.7 % paraformaldehyde in PBS for 20 min at room temperature, and then washed with PBS 5 times. The cover slips were mounted in Dako fluorescent mounting medium, put onto slides, and then visualized using a Nikon Optiphot 2 fluorescent microscope (Excitation: 440-505 nm and Emission: 505-585 nm). Image taken by fluorescence microscope showed a successful gene transfection.

1: A composition for use as a pharmaceutical agent delivery carrier comprising of nanoparticles of a cyclic tetrapyrrolic compound and a pharmaceutical agent.

2: A composition of claim 1 wherein said cyclic tetrapyrrolic compound is a compound has formula I, II, III or IV: 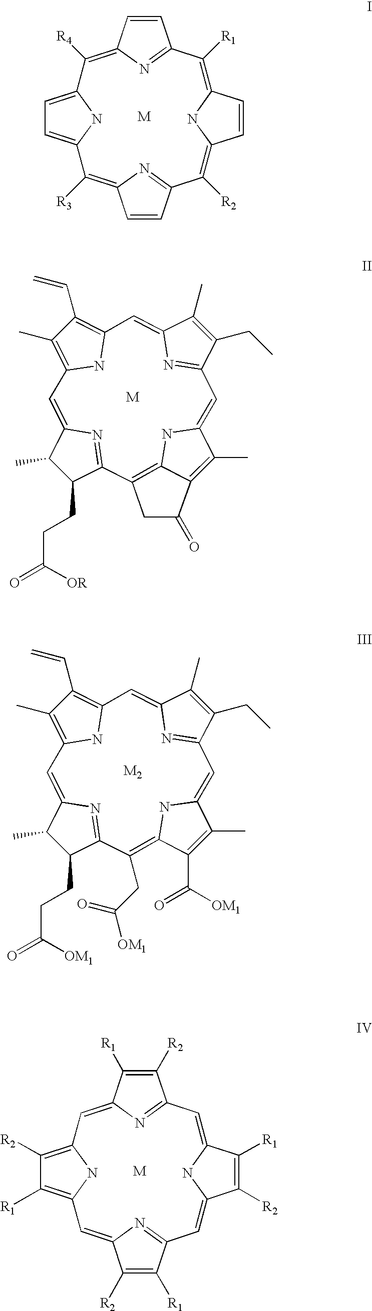
Wherein:
In formula I, R₁, R₂, R₃ and R₄ are substitutes on the porphyrin ring and M is 2H⁺ or a metal ion selected from ions of a group of metals consisting of Mg, Fe, Mn, Co, Ni, Cu, Zn, Sn, Cr, V, Ru, Pt or Pd.
In formula II, R is a substitute and M is 2H⁺ or a metal ion selected from ions of a group of metals consisting of Mg, Fe, Mn, Co, Ni, Cu, Zn, Sn, Cr, V, Ru, Pt or Pd.
In formula III, M₂ is 2H⁺ or a metal ion selected from ions of a group of metals consisting of Mg, Fe, Mn, Co, Ni, Cu, Zn, Sn, Cr, V, Ru, Pt or Pd. M₁ is H⁺ or a metal ion selected from ions of a group of metals consisting of Li, Na, or K.
In formula IV, R₁ and R₂ are substitutes and M is 2H⁺ or a metal ion selected from ions of a group of metals consisting of Mg, Fe, Mn, Co, Ni, Cu, Zn, Sn, Cr, V, Ru, Pt or Pd.

3: A composition of claim 1 wherein said pharmaceutical agent is a nucleic acid.

4: A composition of claim 3 wherein said nucleic acid is a DNA

5: A composition of claim 3 wherein said nucleic acid is a RNA

6: A composition of claim 1 wherein said pharmaceutical agent is a peptide, or a protein, or a non-soluble drug molecule.

7: A composition of claim 2, wherein R is

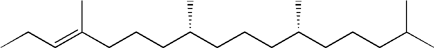

8: A composition of claim 1 wherein said the cyclic tetrapyrrolic compound is chlorophyll a

9: A composition of claim 1 wherein said the cyclic tetrapyrrolic compound is a compound has formula V, VI or VII: 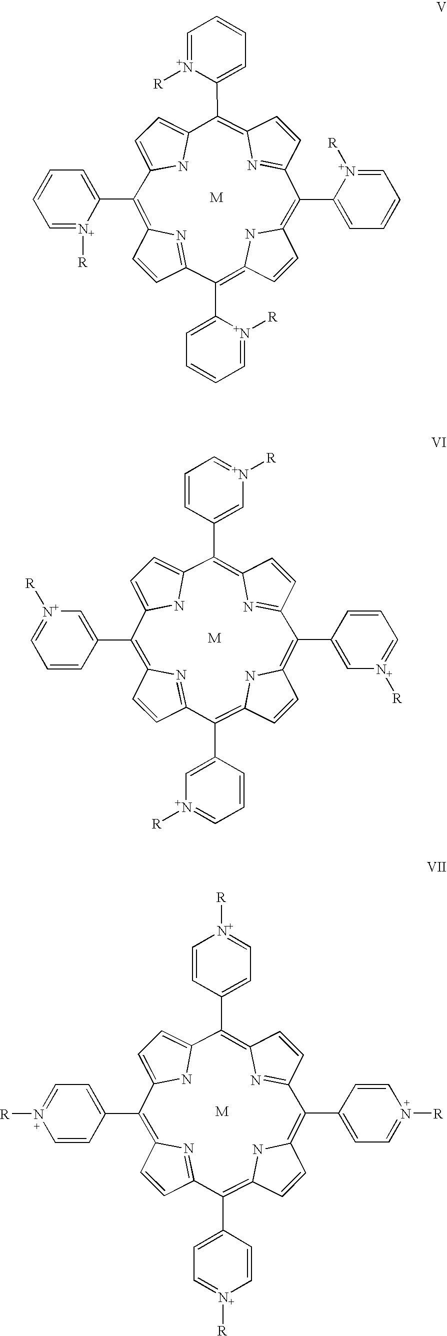
Wherein: R is substitute on the porphyrin ring and M is 2H⁺ or a metal ion selected from ions of a group of metals consisting of Mg, Fe, Mn, Co, Ni, Cu, Zn, Sn, Cr, V, Ru, Pt or Pd.

10: A composition of claim 9 wherein R is —CₙH₂ₙ₊₁, n=1-10

11: A composition of claim 9 wherein said R is —(CₘH₂ₘ)ⱼ(OCₘH₂ₘ)ₚOR, m, j, p=1-10.

12: A method of delivering a pharmaceutical agent into a cell in vivo comprising the steps of:
a. Loading a selected pharmaceutical agent into nanoparticles of a cyclic tetrapyrrolic compound.
b. Contacting the selected cell with the complex under conditions to maintain the viability of the cell

13: A composition of claim 12 wherein said cyclic tetrapyrrolic compound is a compound has formula I, II, III or IV: 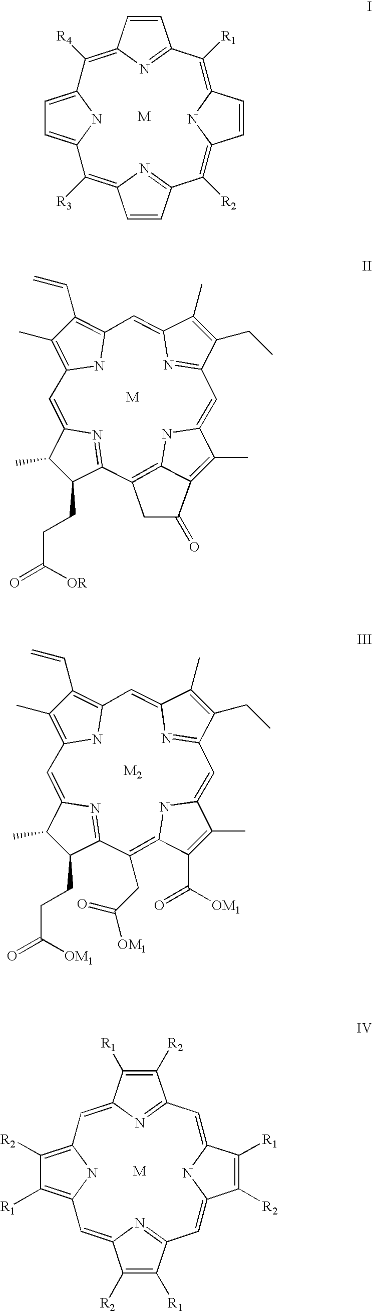
Wherein:
In formula I, R₁, R₂, R₃ and R₄ are substitutes on the porphyrin ring and M is 2H⁺ or a metal ion selected from ions of a group of metals consisting of Mg, Fe, Mn, Co, Ni, Cu, Zn, Sn, Cr, V, Ru, Pt or Pd.
In formula II, R is a substitute and M is 2H⁺ or a metal ion selected from ions of a group of metals consisting of Mg, Fe, Mn, Co, Ni, Cu, Zn, Sn, Cr, V, Ru, Pt or Pd.
In formula III, M₂ is 2H⁺ or a metal ion selected from ions of a group of metals consisting of Mg, Fe, Mn, Co, Ni, Cu, Zn, Sn, Cr, V, Ru, Pt or Pd. M₁ is H⁺ or a metal ion selected from ions of a group of metals consisting of Li, Na, or K.
In formula IV, R₁ and R₂ are substitutes and M is 2H⁺ or a metal ion selected from ions of a group of metals consisting of Mg, Fe, Mn, Co, Ni, Cu, Zn, Sn, Cr, V, Ru, Pt or Pd.

14: A composition of claim 12 wherein said pharmaceutical agent is a nucleic acid.

15: A composition of claim 14 wherein said nucleic acid is a DNA

16: A composition of claim 14 wherein said nucleic acid is a RNA

17: A composition of claim 12 wherein said pharmaceutical agent is a peptide, or a protein, or a non-soluble drug molecule.

18: A composition of claim 13, wherein R is

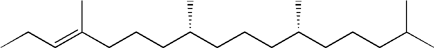

19: A composition of claim 12 wherein said the cyclic tetrapyrrolic compound is chlorophyll a

20: A composition of claim 12 wherein said the cyclic tetrapyrrolic compound is a compound has formula V, VI or VII: 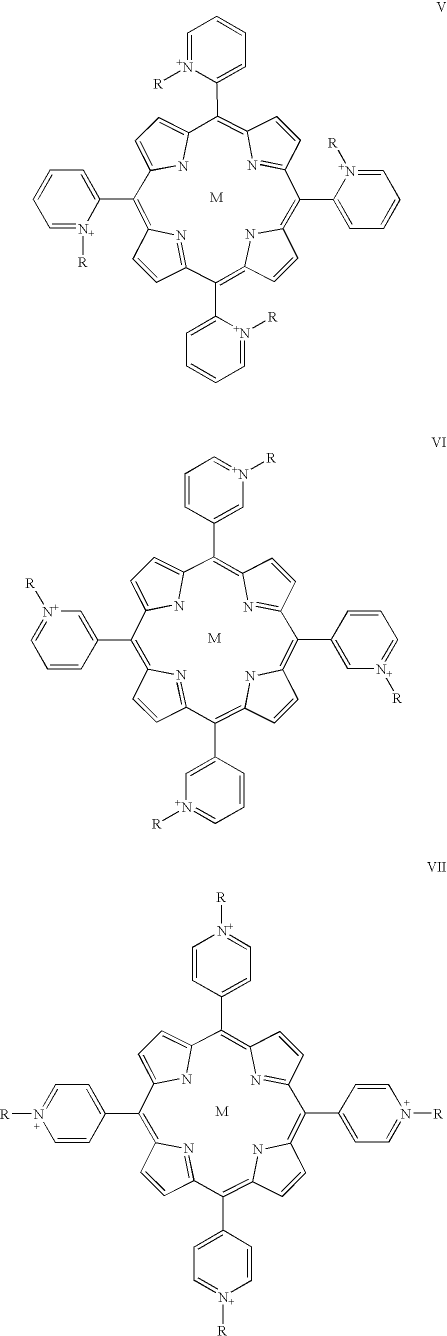
Wherein: R are substitutes on the porphyrin ring and M is 2H⁺ or a metal ion selected from ions of a group of metals consisting of Mg, Fe, Mn, Co, Ni, Cu, Zn, Sn, Cr, V, Ru, Pt or Pd.

21: A composition of claim 20 wherein R is —CₙH₂ₙ₊₁, n=1-10

22: A composition of claim 20 wherein R is —(CₘH₂ₘ)ⱼ(OCₘH₂ₘ)ₚOR, m, j, p=1-10.

23: A composition of claim 2 wherein said substitutes of formula I have at least one cationic groups and one non-polar group.

23: A composition of claim 13 wherein said substitutes of formula I have at least one cationic groups and one non-polar group.
